(19) United States
(12) Patent Application Publication
(10) Pub. No.: US 2012/0157947 A1
(43) Pub. Date: Jun. 21, 2012

(54) Attachment Means For A Conductor-Less Detection System For An Absorbent Article

(76) Inventors: Davis-Dang Hoang Nhan, Appleton, WI (US); Sudhanshu Gakhar, Neenah, WI (US); Katie Josephine Kraus, Menasha, WI (US); Andrew Mark Long, Appleton, WI (US)

(21) Appl. No.: 12/968,399

(22) Filed: Dec. 15, 2010

(51) Int. Cl. A61F 13/42

(52) U.S. Cl. 604/361

(57) ABSTRACT

A wetness sensing system for use with an absorbent article having an inner surface and an outer surface includes a reusable signaling device adapted to sense wetness without the use of conductors within the absorbent article, and a holding fixture adapted to be attached to the inner surface or the outer surface of the absorbent article by a consumer, wherein the signaling device is configured to be coupled to the holding fixture such that the signaling device is positioned adjacent the inner surface or the outer surface of the absorbent article.

### BACKGROUND

Absorbent articles such as diapers, training pants, incontinence products, feminine hygiene products, swim undergarments, and the like, conventionally include a liquid permeable body-side liner, a liquid impermeable outer cover, and an absorbent structure. The absorbent structure is typically located between the outer cover and the liner for taking in and retaining liquids (e.g., urine) exuded by the wearer. The absorbent structure can be made of, for instance, superabsorbent particles. Many absorbent articles, especially those sold under the trade name HUGGIES by the Kimberly-Clark Corporation, are so efficient at absorbing liquids that it is sometimes difficult to tell whether or not the absorbent article has been insulted with a body exudate, especially when the absorbent article is being worn by a newborn or other very young wearers. Insult amounts in such wearers tend to be very small. Other wearers might also produce very small insults.

Accordingly, various types of moisture or wetness indicators have been suggested for use in absorbent articles. The wetness indicators include various passive indicators such as indicator strips, printing, or other devices within each absorbent article, requiring a caregiver to pay for the wetness indicator in each absorbent article whether or not the caregiver intends to use the wetness indicator. Wetness indicators can also include alarm devices that are designed to assist parents or attendants in identifying a wet absorbent article condition early on. The devices can produce an audible, tactile, electromagnetic, or visual signal. Many of these devices rely on electronics, including conductive elements within each absorbent article that can increase the expense of the absorbent article.

In some aspects, for instance, conductive threads or foils have been placed in the absorbent articles that extend from the front of the article to the back of the article. The conductive materials serve as conductive leads for a signaling device and form an open circuit in the article that can be closed when a body fluid, such as urine, closes the circuit.

Incorporating conductive leads into absorbent articles, however, has caused various problems. For example, absorbent articles are typically mass produced on very fast moving machinery. Incorporating conductive leads into an absorbent article at conventional machine speeds has been problematic.

In addition, packaged absorbent articles are typically passed through a metal detector to ensure that there are no foreign objects contained in the package. If the conductive leads are made from or contain a metal, the metal detector can be activated registering a false positive. The incorporation of metallic materials into absorbent articles can also cause problems for those wearing the garments when attempting to pass through security gates that include metal detectors.

In view of the above, a need currently exists for a signaling system for an absorbent article that does not require conductive elements containing metal or other devices to be inserted into the interior of the article.

### SUMMARY

The present inventors undertook intensive research and development efforts with respect to improving absorbent articles, particularly in providing a wetness indicator only when desired by a caregiver and without adding to the cost of an absorbent article. A need exists for wetness detection in absorbent articles and incontinence products in general. Technology that can be implemented without altering absorbent article construction is preferred.

The present disclosure is generally directed to various signaling systems that are particularly well suited for use in conjunction with absorbent articles. The signaling systems, for instance, can be connected to a signaling device that can be configured to emit a signal, such as an audible, tactile, electromagnetic or visual signal, for indicating to a user that a body fluid is present in the absorbent article. For example, in one aspect, the absorbent article includes a diaper and the signaling system is configured to indicate the presence of urine or a bowel movement. In other absorbent articles, however, the signaling systems can be configured to indicate the presence of yeast or metabolites.

More particularly, the present disclosure is directed to a wetness sensing system for use with an absorbent article having an inner surface and an outer surface, the system including a reusable signaling device adapted to sense wetness without the use of conductors within the absorbent article, and a holding fixture adapted to be attached to the inner surface or the outer surface of the absorbent article by a consumer, wherein the signaling device is configured to be coupled to the holding fixture such that the signaling device is positioned adjacent the inner surface or the outer surface of the absorbent article.

The present disclosure is also directed to a wetness sensing system for use with an absorbent article having an outer surface and an inner surface, the system including a signaling device adapted to sense wetness without the use of conductors within the absorbent article, wherein the signaling device is configured to detect wetness using capacitance or inductance, and a holding fixture adapted to be attached to the inner surface or the outer surface of the absorbent article by a consumer, the holding fixture including flexible arms having proximal and distal ends, each distal end including an attachment means, wherein the signaling device is coupled to the holding fixture such that the signaling device is positionable adjacent the inner surface or the outer surface of the absorbent article.

Other features and aspects of the present disclosure are discussed in greater detail herein.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a rear perspective view of one aspect of an absorbent article;

FIG. 2 is a front perspective view of the absorbent article illustrated in FIG. 1 including one aspect of a wetness indicator of the present disclosure;

FIG. 3 is a plan view of the absorbent article shown in FIG. 1 with the article in an unfastened, unfolded and laid flat condition showing the surface of the article that faces away from the wearer;

FIG. 4 is a plan view similar to FIG. 3 showing the surface of the absorbent article that faces the wearer when worn and with portions cut away to show underlying features;

FIG. 5 is a schematic view of one aspect of a wetness sensor system of the present disclosure in association with the absorbent article of FIG. 1;

FIG. 6 is a schematic cross-section view of a wetness sensor system of FIG. 7;

FIG. 7 is a schematic view of another aspect of a wetness sensor system of the present disclosure in association with the absorbent article of FIG. 1;

FIG. 8 is a schematic view of another aspect of a wetness sensor system of the present disclosure in association with the absorbent article of FIG. 1;

FIG. 9 is a schematic view of another aspect of a wetness sensor system of the present disclosure in association with the absorbent article of FIG. 1;

FIG. 10 is a schematic view of another aspect of a wetness sensor system of the present disclosure in association with the absorbent article of FIG. 1;

FIG. 11 is a schematic view of another aspect of a wetness sensor system of the present disclosure in association with the absorbent article of FIG. 1; and

FIGS. 12a and 12b are cross-section schematic views of another aspect of the wetness sensor system of the present disclosure in association with the absorbent article of FIG. 1.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

It is to be understood by one of ordinary skill in the art that the present discussion is a description of exemplary aspects only, and is not intended as limiting the broader aspects of the present disclosure.

The present disclosure is generally directed to signaling systems for absorbent articles that indicate to a user when a body fluid has insulted the article. For example, in one aspect, the signaling system is designed to emit a signal when urine is detected in the absorbent article. Of particular advantage, signaling systems made in accordance with the present disclosure can sense the presence of a body fluid within the absorbent article without having to construct the absorbent article with any elements or sensors contained in the interior of the article. In the past, for instance, metallic conductive leads were typically placed within the interior of the absorbent article. The signaling systems of the present disclosure, on the other hand, can sense the presence of a body fluid from an exterior surface of the article that can greatly simplify the incorporation of the signaling system into the article.

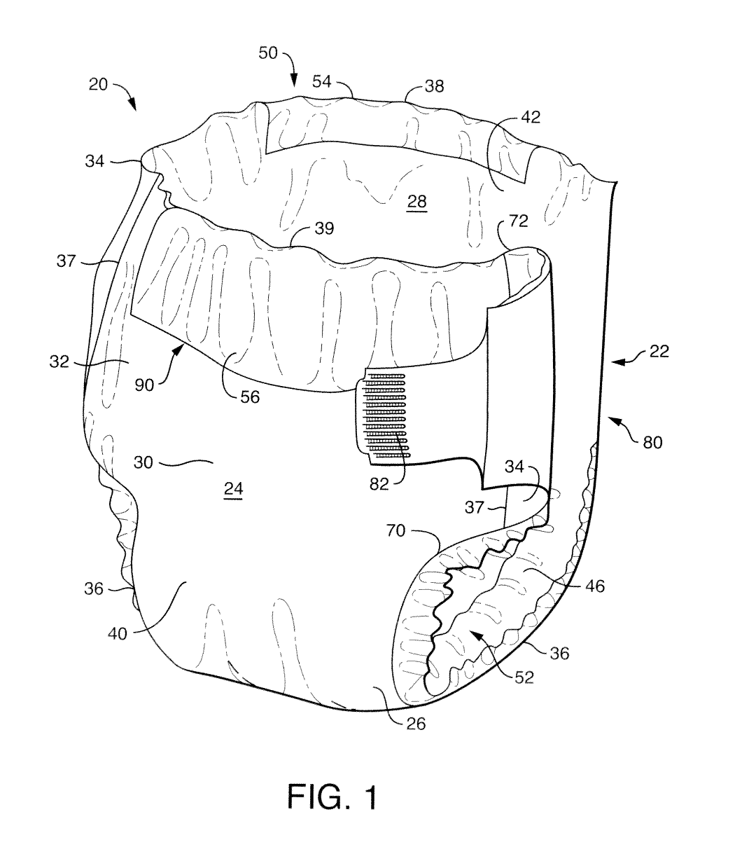

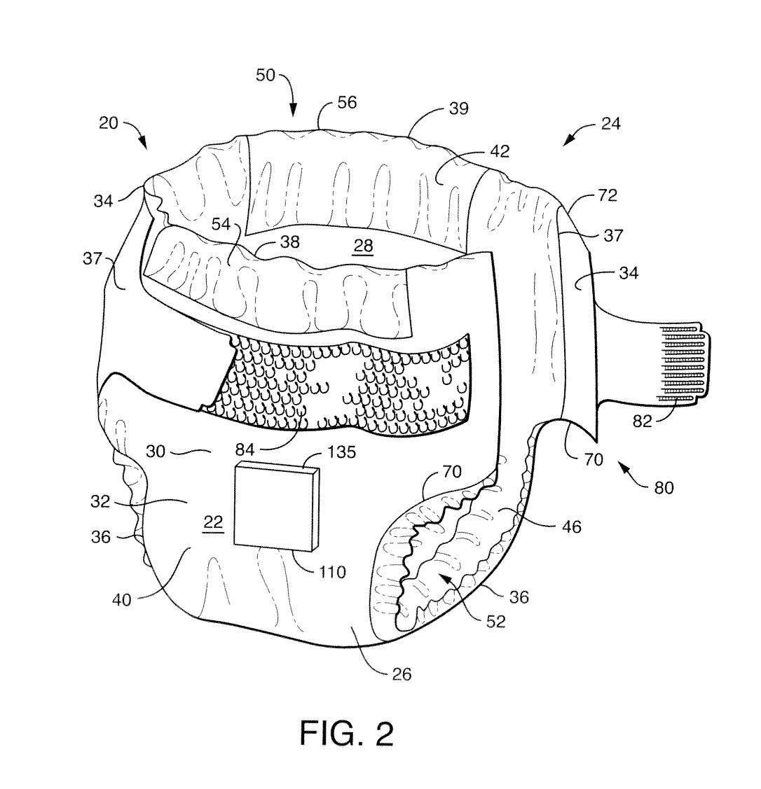

In accordance with the present disclosure, the signaling system can have various configurations and designs. Referring to FIGS. 1 and 2, for exemplary purposes, an absorbent article 20 that can be used in conjunction with signaling systems of the present disclosure is shown. The absorbent article 20 can be disposable or not. It is understood that the present disclosure is suitable for use with various other absorbent articles intended for personal wear, including but not limited to diapers, training pants, swim pants, feminine hygiene products, incontinence products, medical garments, surgical pads and bandages, other personal care or health care garments, and the like, without departing from the scope of the present disclosure.

By way of illustration only, various materials and methods for constructing absorbent articles such as the absorbent article 20 of the various aspects of the present disclosure are disclosed in PCT Patent Disclosure WO 00/037009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

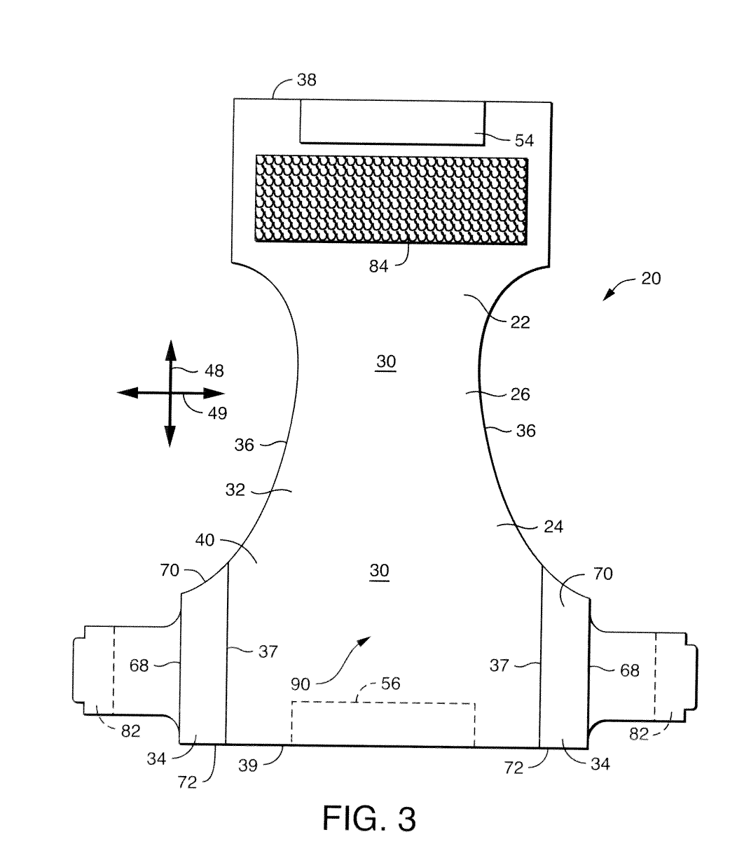

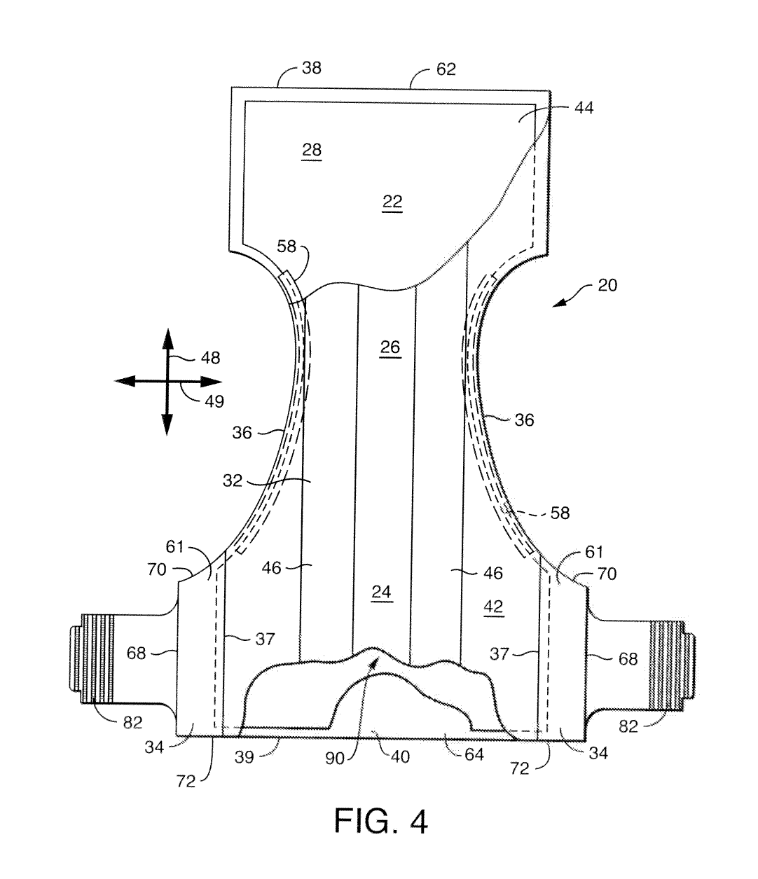

An absorbent article 20 is representatively illustrated in FIG. 1 in a partially fastened condition. The absorbent article 20 shown in FIGS. 1 and 2 is also represented in FIGS. 3 and 4 in an opened and unfolded state. Specifically, FIG. 3 is a plan view illustrating the exterior side of the absorbent article 20, while FIG. 4 illustrates the interior side of the absorbent article 20. As shown in FIGS. 3 and 4, the absorbent article 20 defines a longitudinal direction 48 that extends from the front of the article, when worn, to the back of the article. Orthogonal to the longitudinal direction 48 is a transverse direction 49.

The absorbent article 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The absorbent article 20 also defines an inner surface 28 adapted in use (e.g., positioned relative to the other components of the article 20) to be disposed toward the wearer, and an outer surface 30 opposite the inner surface 28. The front and back regions 22, 24 are those portions of the absorbent article 20, that, when worn, wholly or partially cover or encircle the waist or mid-lower torso of the wearer. The crotch region 26 generally is that portion of the absorbent article 20 that, when worn, is positioned between the legs of the wearer and covers the lower torso and crotch of the wearer. The absorbent article 20 has a pair of laterally opposite side edges 36 and a pair of longitudinally opposite waist edges, respectively designated front waist edge 38 and back waist edge 39.

The illustrated absorbent article 20 includes a chassis 32 that, in this aspect, encompasses the front region 22, the back region 24, and the crotch region 26. Referring to FIGS. 1-4, the chassis 32 includes an outer cover 40 and a bodyside liner 42 (FIGS. 1 and 4) that can be joined to the outer cover 40 in a superimposed relation therewith by adhesives, ultrasonic bonds, thermal bonds or other conventional techniques. Referring to FIG. 4, the liner 42 can suitably be joined to the outer cover 40 along the perimeter of the chassis 32 to form a front waist seam 62 and a back waist seam 64. As shown in FIG. 4, the liner 42 can suitably be joined to the outer cover 40 to form a pair of side seams 61 in the front region 22 and the back region 24. The liner 42 can be generally adapted, i.e., positioned relative to the other components of the article 20, to be disposed toward the wearer's skin during wear of the absorbent article 20. The chassis 32 can further include an absorbent structure 44 particularly shown in FIG. 4 disposed between the outer cover 40 and the bodyside liner 42 for absorbing liquid body exudates exuded by the wearer, and can further include a pair of containment flaps 46 secured to the bodyside liner 42 for inhibiting the lateral flow of body exudates.

The elasticized containment flaps 46 as shown in FIG. 4 define a partially unattached edge that assumes an upright configuration in at least the crotch region 26 of the absorbent article 20 to form a seal against the wearer's body. The containment flaps 46 can extend longitudinally along the entire length of the chassis 32 or can extend only partially along the length of the chassis 32. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the absorbent article 20 can also suitably include leg elastic members 58 (FIG. 4), as are known to those skilled in the art. The leg elastic members 58 can be operatively joined to the outer cover 40 and/or the bodyside liner 42 and positioned in the crotch region 26 of the absorbent article 20.

The leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example, with the disclosure of heat, such that elastic retractive forces are imparted to the substrate. In one particular aspect, for example, the leg elastic members 58 can include a plurality of dry-spun coalesced multifilament spandex elastomeric threads sold under the trade name LYCRA and available from Invista, Wilmington, Del., U.S.A.

In some aspects, the absorbent article 20 can further include a surge management layer (not shown) that can be optionally located adjacent the absorbent structure 44 and attached to various components in the article 20 such as the absorbent structure 44 or the bodyside liner 42 by methods known in the art, such as by using an adhesive. A surge management layer helps to decelerate and diffuse surges or gushes of liquid that can be rapidly introduced into the absorbent structure 44 of the article 20. Desirably, the surge management layer can rapidly accept and temporarily hold the liquid prior to releasing the liquid into the storage or retention portions of the absorbent structure 44. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

As shown in FIGS. 1-4, the absorbent article 20 further includes a pair of opposing elastic side panels 34 that are attached to the back region of the chassis 32. As shown particularly in FIGS. 1 and 2, the side panels 34 can be stretched around the waist and/or hips of a wearer to secure the garment in place. As shown in FIGS. 3 and 4, the elastic side panels 34 are attached to the chassis 32 along a pair of opposing longitudinal edges 37. The side panels 34 can be attached or bonded to the chassis 32 using any suitable bonding technique. For instance, the side panels 34 can be joined to the chassis 32 by adhesives, ultrasonic bonds, thermal bonds, or other conventional techniques.

In an alternative aspect, the elastic side panels 34 can also be integrally formed with the chassis 32. For instance, the side panels 34 can include an extension of the bodyside liner 42, of the outer cover 40, or of both the bodyside liner 42 and the outer cover 40.

In the aspects shown in the figures, the side panels 34 are connected to the back region 24 of the absorbent article 20 and extend over the front region 22 of the article 20 when securing the article 20 in place on a user. It should be understood, however, that the side panels 34 can alternatively be connected to the front region 22 of the article 20 and extend over the back region 24 when the article 20 is donned.

With the absorbent article 20 in the fastened position as partially illustrated in FIGS. 1 and 2, the elastic side panels 34 can be connected by a fastening system 80 to define a 3-dimensional absorbent article configuration having a waist opening 50 and a pair of leg openings 52. The waist opening 50 of the article 20 is defined by the waist edges 38 and 39 that encircle the waist of the wearer.

In the aspects shown in the figures, the side panels 34 are releasably attachable to the front region 22 of the article 20 by the fastening system 80. It should be understood, however, that in other aspects, the side panels 34 can be permanently joined to the chassis 32 at each end. The side panels 34 can be permanently bonded together, for instance, when forming a training pant or absorbent swimwear.

The elastic side panels 34 each have a longitudinal outer edge 68, a leg end edge 70 disposed toward the longitudinal center of the absorbent article 20, and waist end edges 72 disposed toward a longitudinal end of the absorbent article 20. The leg end edges 70 of the absorbent article 20 can be suitably curved and/or angled relative to the lateral direction 49 to provide a better fit around the wearer's legs. However, it is understood that only one of the leg end edges 70 can be curved or angled, such as the leg end edge 70 of the back region 24, or alternatively, neither of the leg end edges 70 can be curved or angled, without departing from the scope of the present disclosure. As shown in FIG. 4, the outer edges 68 are generally parallel to the longitudinal direction 48 while the waist end edges 72 are generally parallel to the transverse axis 49. It should be understood, however, that in other aspects the outer edges 68 and/or the waist edges 72 can be slanted or curved as desired. Ultimately, the side panels 34 are generally aligned with a waist region 90 of the chassis 32.

The fastening system 80 can include laterally opposite first fastening components 82 adapted for refastenable engagement to corresponding second fastening components 84. In the aspect shown in the figures, the first fastening component 82 is located on the elastic side panels 34, while the second fastening component 84 is located on the front region 22 of the chassis 32. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the article 20 in its three-dimensional configuration.

The fastening components 82, 84 can be any refastenable fasteners suitable for absorbent articles, such as adhesive fasteners, cohesive fasteners, mechanical fasteners, or the like. In particular aspects, the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include hook fasteners and the second fastening components 84 include complementary loop fasteners. Alternatively, the first fastening components 82 can include loop fasteners and the second fastening components 84 can be complementary hook fasteners. In another aspect, the fastening components 82, 84 can be interlocking similar surface fasteners, or adhesive and cohesive fastening elements such as an adhesive fastener and an adhesive-receptive landing zone or material; or the like.

In addition to possibly having elastic side panels 34, the absorbent article 20 can include various waist elastic members for providing elasticity around the waist opening. For example, as shown in the figures, the absorbent article 20 can include a front waist elastic member 54 and/or a back waist elastic member 56.

The absorbent article 20 can include a wetness sensing system 110 that can be used to detect wetness, such as from urine, feces, or other body exudates, within the absorbent article 20. The wetness sensing system 110 includes a reusable signaling device 120 and a holding fixture 150.

The reusable signaling device 120 is adapted to sense wetness without the use of conductors within the absorbent article 20. Suitable wetness sensing technologies include capacitance-based sensors such as those described in co-pending U.S. patent Ser. No. 12/648,645, inductance-based sensors such as those described in co-pending U.S. patent Ser. No. 11/983,970, and infrared reflectance-based sensors such as those described in co-pending U.S. patent Ser. No. 12/636,888, each of which is incorporated herein by reference to the extent it does not conflict herewith. Any other suitable sensor technology can be employed.

Sensors such as those described herein are further described, for instance, in U.S. Patent Disclosure Publication No. 2008/0048786, which is incorporated herein by reference to the extent it does not conflict herewith.

The signaling device 120 can include a housing 125. The housing 125 can be designed so that the signaling device 120 minimizes any discomfort when worn. In addition to being comfortable when attached to an absorbent article 20, however, the signaling device 120 must also be durable. Based upon anthropomorphic modeling, signaling devices 120 made in accordance with the present disclosure are produced with dimensions that fall within a set of desirable ranges. In addition, the signaling device 120 can also include radii on the corners of the signaling device 120 that are constrained by a set of desirable ranges to ensure that the corners do not impinge, scratch, or poke the body's surface.

In this aspect, the signaling device 120 can include a housing 125 that contains the internal components. The internal components, for instance, can include a battery and can be configured to generate an audible signal, a tactile signal, an electromagnetic signal, a wireless signal, a visual signal, any other suitable signal, or any combination of these. The housing 125 in this aspect can have a rectangular shape, such as a square shape, with rounded corners, or the housing 125 can be circular, ovoid, or any other suitable shape. Housings 125 suitable for use in the wetness sensing system 110 described herein include those further described, for instance, in U.S. patent Ser. No. 11/848,714, which is incorporated herein by reference to the extent it does not conflict herewith.

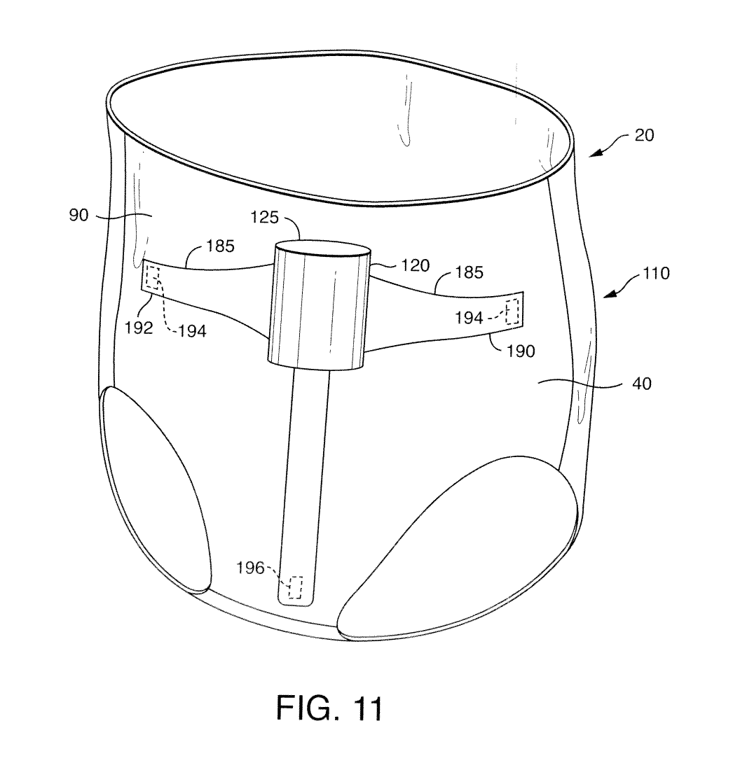

As illustrated in FIG. 11, the signaling device 120 can also include a flexible sensor band 130 extending from the housing 125. The flexible sensor band 130 is a flexible plastic film including an array of capacitive-based sensing elements. The sensing elements can take form of two planar electrodes making an open-face virtual capacitor. The flexible sensor band 130 can be made by etching from copper sheets laminated onto a flexible and non-conductive substrate. Such flexible and non-conductive substrates include polyimide, polyester, and any other suitable material. An example of such material is KAPTON films made by E. I. du Pont de Nemours and Company of Wilmington, Del. The etching method is widely used to make printed circuit boards in the electronic industry. Other methods of making a flexible printed circuit board include silk screen printing, gravure printing, and flexographic printing. The conductive material forming open face capacitors is made with conductive materials such as copper, silver, or carbon black. The flexible sensing band 130 can be electrically connected to the signaling device 120 by a ZIF connector or any other suitable connector.

As stated above, the wetness sensing system 110 includes a holding fixture 150. The signaling device 120 is attached to the holding fixture 150 by either a manufacturer or by a consumer. The holding fixture 150 can be part of the housing 125 when the housing 125 is made. In other aspects, the holding fixture 150 can be attached to the housing 125 by injection molding, stitching, adhesive bonding, ultrasonic bonding or thermal bonding. The holding fixture 150 can also be attached to the housing 125 by a mechanical attachment mean, such as nut and bolt, snap-on or screw-on configurations, or any other suitable attachment means.

The holding fixture 150 is adapted to be attached to the inner surface 28 or the outer surface 30 of the absorbent article 20 by a consumer. The holding fixture 150 is attached to the inner surface 28 or the outer surface 30 such that the signaling device 120 is positioned adjacent the inner surface 28 or the outer surface 30 of the absorbent article 20.

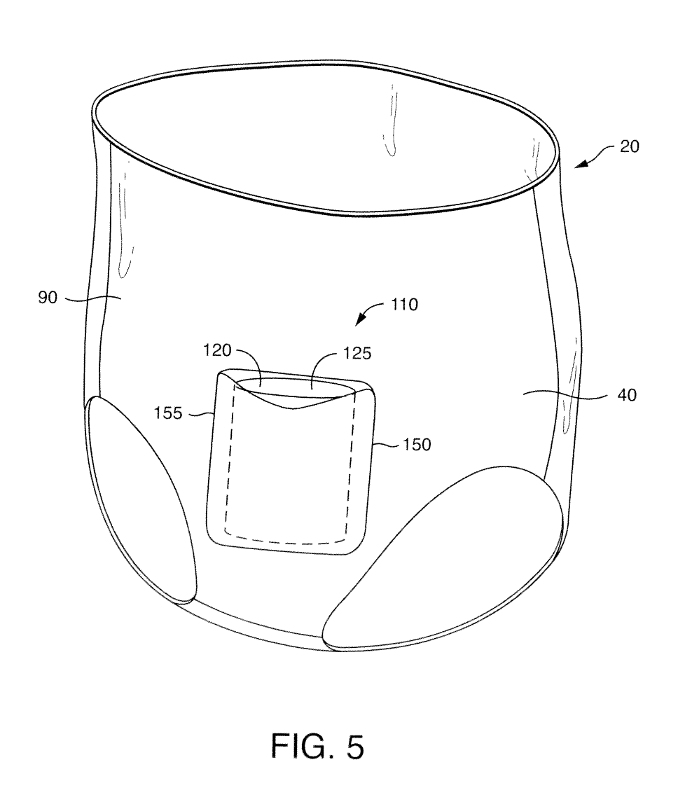

In one aspect of the present disclosure illustrated in FIG. 5, the holding fixture 150 includes a pocket 155. The pocket 155 is attachable to the inner surface 28 or the outer surface 30 by the consumer. The pocket 155 can be attachable to the inner surface 28 or the outer surface 30 using adhesives, cohesives, mechanical fasteners such as hook material, or any other suitable attachment means. In one particular aspect, the pocket 155 can be attached to the inner surface 28 or the outer surface 30 with a pressure sensitive adhesive. The pocket 155 is attached to the inner surface 28 or the outer surface 30 with a removal peel strength of at least 800 g/inch. Once the consumer attaches the pocket 155 to the inner surface 28 or the outer surface 30, the consumer can position the signaling device 120 within the pocket 155. The signaling device 120 is then ready to sense wetness within the article 20. The signaling device 120 is removed from the pocket 155 after article usage. The pocket 155 is then disposed with the article 20. The pocket 155 can be manufactured from woven material or non-woven material such as SMS, spun-bond, or film such as polypropylene or the like, or from any other suitable material, either alone or laminated to a non-woven.

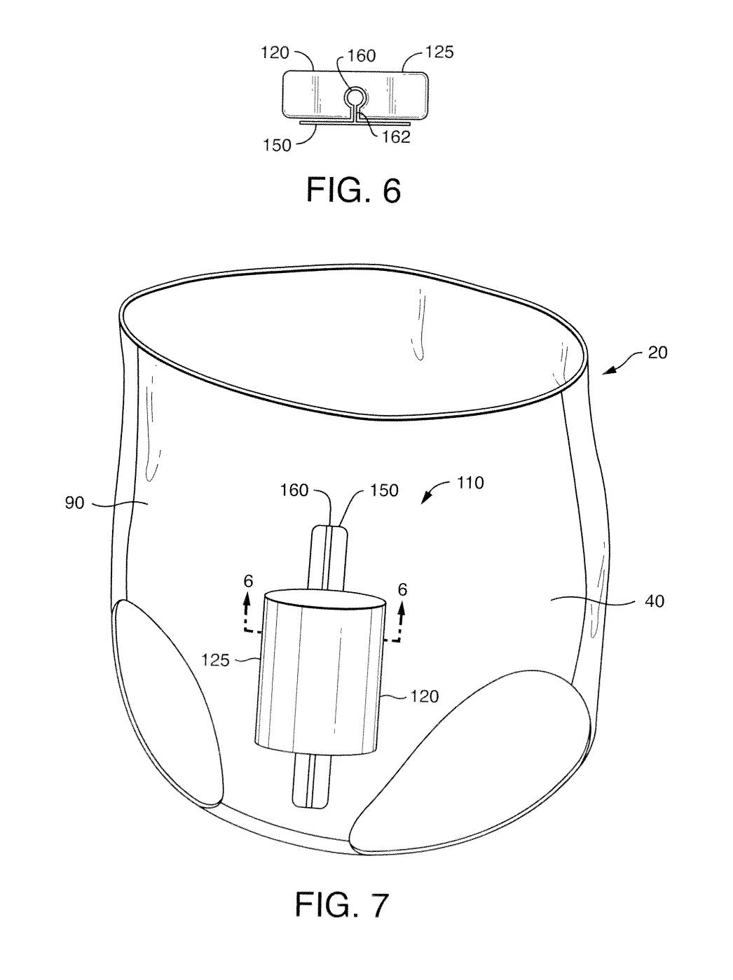

In another aspect of the present disclosure illustrated in FIGS. 6 and 7, the holding fixture 150 includes a rail 160. The rail 160 is attachable to the inner surface 28 or the outer surface 30 by the consumer. The rail 160 can be attachable to the inner surface 28 or the outer surface 30 using adhesives, cohesives, mechanical fasteners such as hook material, or any other suitable attachment means. In this aspect, the signaling device 120 includes a slot 162 or other structure sized to accommodate the rail 160. The consumer can attach the rail 160 to the inner surface 28 or the outer surface 30. The consumer can then attach the signaling device 120 to the rail 160 by positioning the slot 162 on the rail 160. The signaling device 120 is then ready to sense wetness within the article 20. The rail 160 can also be attached as a part of the article manufacturing process.

The rail 160 can be a flexible, extruded plastic part. The rail 160 can also be manufactured from non-woven material such as SMS, spun-bond, or film such as polypropylene or the like, either as an individual component or as a laminate/combination of materials. The rail 160 can also be a gathered zone of facing, outer cover laminate, or product constituent such as liner or absorbent core. The rail 160 can also be added as a graspable rail attached to the inner surface 28 or the outer surface 30 externally or underneath layers.

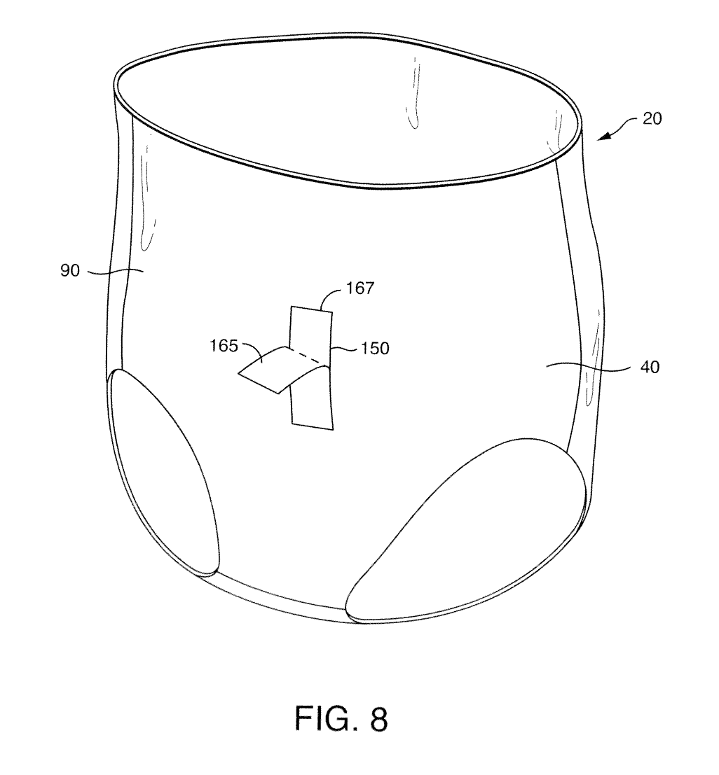

In still another aspect of the present disclosure illustrated in FIG. 8, the holding fixture 150 includes a tab 165 and strip 167. The strip 167 is attachable to the inner surface 28 or the outer surface 30 by the consumer. The strip 167 can be attachable to the inner surface 28 or the outer surface 30 using adhesives, cohesives, mechanical fasteners such as hook material, or any other suitable attachment means. In a particular aspect, the strip 167 can include a thick plastic but flexible material. A portion of the strip 167 is bonded to the inner surface 28 or the outer surface 30. The tab 165, which is the unbonded portion of the strip 167, is used to anchor the signaling device 120. The strip 167 is disposed with the product after product usage. The article 20 when worn has a curvature through the target zone, which requires the strip material to be flexible and move with the article 20 in use. In this aspect, the signaling device 120 includes a clamp or other suitable structure sized to accommodate the tab 165. The consumer can attach the tab 165 to the inner surface 28 or the outer surface 30. The consumer can then attach the signaling device 120 to the tab 165 by positioning the clamp on the tab 165. The signaling device 120 is then ready to sense wetness within the article 20. The tab 165 can also be attached as a part of the article manufacturing process.

In another aspect of the present disclosure, the tab 165 can be a part of a disposal feature. In this aspect, a portion of the tab 165 can be used to attach the signaling device 120, and another portion of the tab 165 can be wrapped around the article 20 for disposal purposes. In a similar aspect of the present disclosure, the tab 165 can be a loop of material. The tab 165 can be manufactured from woven material or non-woven material such as SMS, spun-bond, or film such as polypropylene or the like, or from any other suitable material, either alone or laminated to a non-woven.

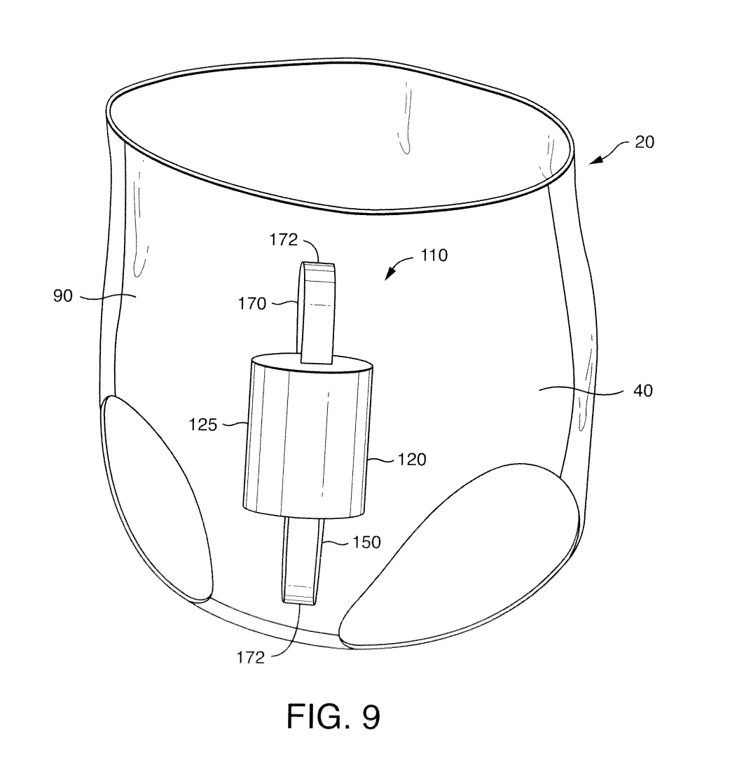

In still another aspect of the present disclosure illustrated in FIG. 9, the holding fixture 150 includes a strap 170. The strap 170 is attachable to the inner surface 28 or the outer surface 30 by the consumer. The strap 170 can be attachable to the inner surface 28 or the outer surface 30 using adhesives, cohesives, mechanical fasteners such as hook material, or any other suitable attachment means. In a particular aspect, the strap 170 includes distal ends 172, with the attachment means disposed at the distal ends 172 of the strap 170. Once the consumer attaches the strap 170 to the inner surface 28 or the outer surface 30 or other structure of the article 20, the consumer can then attach the signaling device 120 to the holding fixture 150. In other aspects, the signaling device 120 is attached to the holding fixture 150 by a manufacturer, and the consumer only needs to attach the holding fixture 150 to the inner surface 28 or the outer surface 30. The signaling device 120 is then ready to sense wetness within the article 20. The strap 170 can be manufactured from woven material or non-woven material such as SMS, spun-bond, or film such as polypropylene or the like, or from any other suitable material, either alone or laminated to a non-woven. The strap 170 can also be used as a disposal feature.

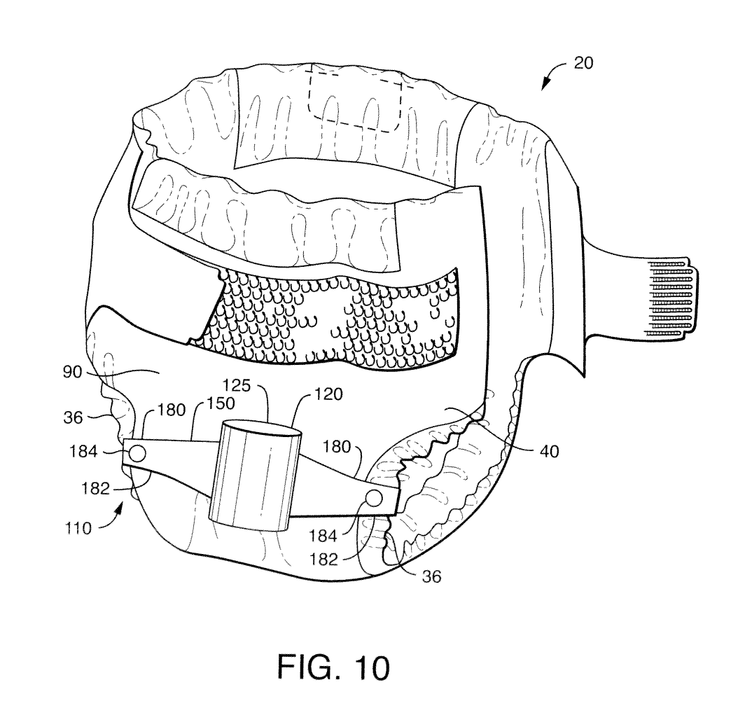

In still another aspect of the present disclosure illustrated in FIG. 10, the holding fixture 150 includes grip arms 180. The grip arms 180 are attachable to the inner surface 28 or the outer surface 30 by the consumer. The grip arms 180 can be attachable to the inner surface 28 or the outer surface 30 using adhesives, cohesives, mechanical fasteners such as hook material, or any other suitable attachment means. In a particular aspect, the grip arms 180 includes distal ends 182, with the attachment means disposed at the distal ends 182 of the grip arms 180. The distal ends 182 of the grip arms 180 can wrap around the side edges 36 of the article 20 and can, if equipped with an attachment means such as a snap mechanism 184, attach to itself through the material of the side edges 36. In another aspect, an attachment means such as tarp-clip-type of fastener can be used, such as one that has ratchet and lock jaw to grab the material tightly. In this aspect, the fastening means can be used to grab the leg opening 52 or a portion of the inner surface 28 or the outer surface 30. Once the consumer attaches the grip arms 180 to the inner surface 28 or the outer surface 30 or other structure of the article 20, the consumer can then attach the signaling device 120 to the holding fixture 150. In other aspects, the signaling device 120 is attached to the holding fixture 150 by a manufacturer, and the consumer only needs to attach the holding fixture 150 to the inner surface 28 or the outer surface 30. The signaling device 120 is then ready to sense wetness within the article 20. The grip arms 180 can be manufactured from woven material or non-woven material such as SMS, spun-bond, or film such as polypropylene or the like, or from any other suitable material, either alone or laminated to a non-woven.

In an alternative aspect of the present disclosure illustrated in FIG. 11, the holding fixture 150 includes flexible arms 185 having proximal and distal ends 190, 192. Each distal end 192 can include an attachment means 194. The flexible arms 185 are attachable to the inner surface 28 or the outer surface 30 by the consumer. The flexible arms 185 can be attachable to the inner surface 28 or the outer surface 30 using adhesives, cohesives, mechanical fasteners such as hook material, or any other suitable attachment means. The flexible sensor band 130, if present, can also include an attachment means 196 such that the flexible sensor band 130 is attachable to the inner surface 28 or the outer surface 30 by the consumer. The flexible sensor band 130 can be attachable to the inner surface 28 or the outer surface 30 using adhesives, cohesives, mechanical fasteners such as hook material, or any other suitable attachment means. Once the consumer attaches the flexible arms 185 and the flexible sensor band 130 to the inner surface 28 or the outer surface 30, the consumer can then attach the signaling device 120 to the holding fixture 150. In other aspects, the signaling device 120 is attached to the holding fixture 150 by a manufacturer, and the consumer only needs to attach the holding fixture 150 to the inner surface 28 or the outer surface 30. The signaling device 120 is then ready to sense wetness within the article 20. The flexible arms 185 and the flexible sensor band 130 can be manufactured from woven material or non-woven material such as SMS, spun-bond, film such as polypropylene, rubber, other elastomeric material, or from any other suitable material, either alone or laminated to a non-woven.

In an alternate aspect of the present disclosure, the flexible arms 185, the flexible band 130, and the signaling device 120 can be formed together as one unit of injection-molded plastic. The attachment means 194, 196 can be an adhesive, a cohesive, hook material, or any other suitable attachment material.

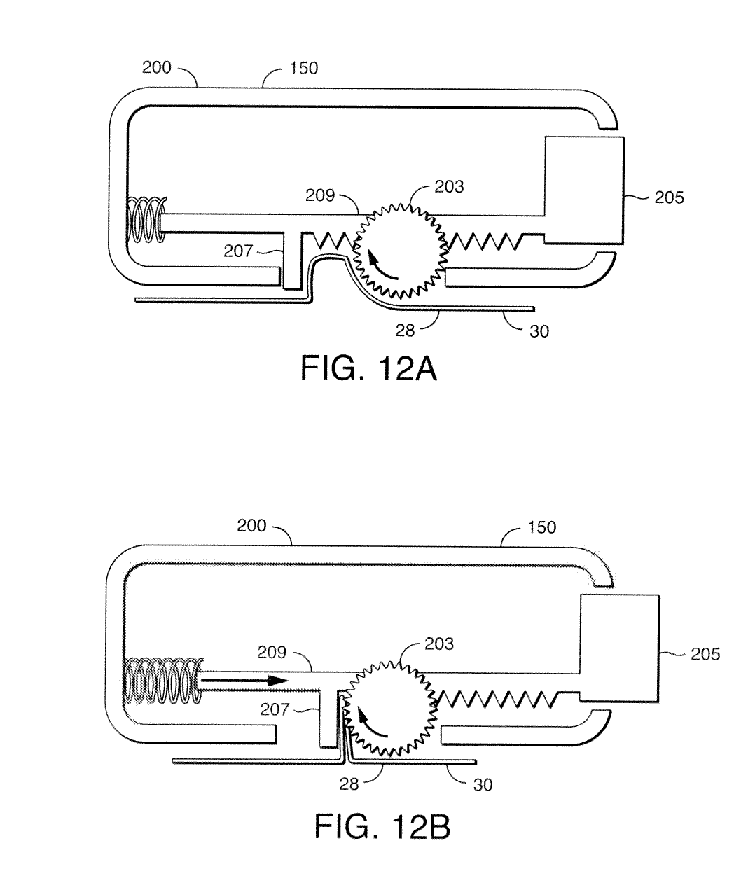

In still another aspect of the present disclosure illustrated in FIGS. 12a and 12b, the holding fixture 150 includes mechanical clamp 200 to grab, pinch, and/or clamp the inner surface 28 or the outer surface 30 of the article 20 by the consumer. An example of a mechanical clamp 200 includes a roller 203 and a spring-loaded arm 209 with a push button 205. The button 205 can be pressed by the consumer as the consumer places the holding fixture 150 against the inner surface 28 or the outer surface 30, as illustrated in FIG. 12a. As the consumer releases the button 205, the roller 203 can gather the material from the article 20. The material is grabbed and pinched between the roller 203 and the stop 207 of the arm 209, as illustrated in FIG. 12b. The signaling device 120 is then ready to sense wetness within the article 20. The roller 203, the arm 209, and the other structures can be manufactured from a plastic material, injection-molded plastics, or from any other suitable material.

Various attachment mechanisms include those disclosed in co-pending and co-assigned U.S. Patent Disclosure Publication No. 2007/0142797 to Long, et al. and entitled “Garments With Easy-To-Use Signaling Device”; U.S. Patent Disclosure Publication No. 2006/0244614 to Long and entitled “Connection Mechanisms”; and U.S. Patent Disclosure Publication No. 2007/0024457 to Long, et al. and entitled “Connection Mechanisms In Absorbent Articles For Body Fluid Signaling Devices,” which are incorporated herein by reference to the extent they are consistent (i.e., not in conflict) herewith.

The holding fixture 150 can be designed to be disposed with the article 20 to which it is attached if generally disposable materials are chosen for the holding fixture 150. Providing a holding fixture 150 that is disposed of with the article 20 can provide improved cleanliness, and ease of use, and can benefit from not needing to be removed from a used article 20. A holding fixture 150 might also be not easy to reattach if used more than once. In these aspects the materials from which the holding fixture 150 is made from should be relatively inexpensive.

The holding fixture 150 can be designed for reuse if generally durable materials are chosen for the holding fixture 150. Providing a reusable holding fixture 150 enhances durability, is more easily washed, and its positioning can be more easily adjusted if a mechanical fastener such as hook material is used instead of an adhesive.

The signaling device 120 can emit any suitable signal to indicate to the user that the absorbent article 20 has been insulted. The signal, for instance, can include an audible signal, a tactile signal, an electromagnetic signal, a wireless signal, a visual signal, any other suitable signal, or any combination of these. The audible signal, for instance, can be as simple as a beep or can include a musical tune. In still another aspect, the signaling device can emit a wireless signal that then activates a remote device, such as a telephone or a pager.

Further aspects of the signaling device 120 can be found in co-pending U.S. patent Ser. No. 12/347,539, entitled “Remote Detection Systems For Absorbent Articles,” which is incorporated herein by reference to the extent it does not conflict herewith.

In an alternative aspect, the signaling device 120 can be configured to be removed from the absorbent article 20 when the absorbent article 20 is disposed and placed on a new absorbent article 20. In fact, in one aspect, the signaling device 120 can include multiple settings depending upon the absorbent article 20 to which it is attached. In this manner, the signaling system can be modified based upon the particular product specifications. The product purchased can provide information to the consumer as to which setting to use.

These and other modifications and variations to the present disclosure can be practiced by those of ordinary skill in the art, without departing from the spirit and scope of the present disclosure, which is more particularly set forth in the appended claims. In addition, it should be understood that elements of the various aspects can be interchanged both in whole and in part. Furthermore, those of ordinary skill in the art will appreciate that the foregoing description is by way of example only, and is not intended to limit the disclosure so further described in such appended claims.

What is claimed:

1. A wetness sensing system for use with an absorbent article having an inner surface and an outer surface, the system comprising:
a reusable signaling device adapted to sense wetness without the use of conductors within the absorbent article; and
a holding fixture adapted to be attached to the inner surface or the outer surface of the absorbent article by a consumer, wherein the signaling device is configured to be coupled to the holding fixture such that the signaling device is positioned adjacent the inner surface or the outer surface of the absorbent article.

2. The system of claim 1, wherein the holding fixture includes a pocket.

3. The system of claim 1, wherein the holding fixture includes a rail, and wherein the signaling device includes a slot sized to accommodate the rail.

4. The system of claim 1, wherein the holding fixture includes flexible arms having proximal and distal ends, each distal end including an attachment means.

5. The system of claim 1, wherein the holding fixture includes a strap.

6. The system of claim 1, wherein the holding fixture includes a tab.

7. The system of claim 1, wherein the signaling device includes a housing and a flexible sensor band extending from the housing.

8. The system of claim 1, wherein the holding fixture includes a mechanical attachment means.

9. The system of claim 8, wherein the mechanical attachment means is hook material.

10. The system of claim 8, wherein the holding fixture includes a mechanical clamp.

11. The system of claim 8, wherein the mechanical attachment means is a snap mechanism.

12. The system of claim 1, wherein the holding fixture includes an adhesive attachment means.

13. The system of claim 1, wherein the signaling device is configured to detect wetness using capacitance.

14. The system of claim 1, wherein the signaling device is configured to detect wetness using inductance.

15. The system of claim 1, wherein the signaling device is configured to detect wetness using infrared reflectance.

16. The system of claim 1, wherein the signaling device is configured to be coupled to the holding fixture by a manufacturer.

17. The system of claim 1, wherein the signaling device is configured to be coupled to the holding fixture by a consumer.

18. The system of claim 1, wherein the holding fixture is adapted to be reused.

19. The system of claim 1, wherein the absorbent article is disposable, and wherein the holding fixture is adapted to be disposable with the disposable absorbent article.

20. A wetness sensing system for use with an absorbent article having an outer surface and an inner surface, the system comprising:
a signaling device adapted to sense wetness without the use of conductors within the absorbent article, wherein the signaling device is configured to detect wetness using capacitance or inductance; and
a holding fixture adapted to be attached to the inner surface or the outer surface of the absorbent article by a consumer, the holding fixture including flexible arms having proximal and distal ends, each distal end including an attachment means, wherein the signaling device is coupled to the holding fixture such that the signaling device is positionable adjacent the inner surface or the outer surface of the absorbent article.

21. The system of claim 20, wherein the attachment means is hook material.

22. The system of claim 20, wherein the signaling device includes a housing and a flexible sensor band extending from the housing.
